(19) United States
(12) Patent Application Publication
(10) Pub. No.: US 2010/0292641 A1
(43) Pub. Date: Nov. 18, 2010

(54) Targeted drug delivery device and method

(76) Inventors: Bandula Wijay, Friendswood, TX (US); Nandhika Wijay, Friendswood, TX (US)

Correspondence Address: KAJANE MCMANUS, 550 MARINA PARKWAY, SUITE E2, PMB 88, CHULA VISTA, CA, 91910, US

(21) Appl. No.: 12/586,033

(22) Filed: Sep. 16, 2009

Related U.S. Application Data
(60) Provisional application No. 61/216,331, filed on May 15, 2009.

(51) Int. Cl. A61M 25/10

(52) U.S. Cl. 604/103.02

(57) ABSTRACT

A catheter described is a balloon angioplasty catheter either of Over the Wire design or the Rapid Exchange design and has an expansible sleeve over the balloon. The expansible sleeve is porous and is able to absorb large quantities of fluid and fluids containing anti-proliferative drugs or other bioactive agents. The porous sleeve also expands along with the balloon when the balloon is inflated making the porous sleeve in contact with the inner wall of the affected artery or body cavity. The pressure in the balloon can be increased or pulsed, using a piezoelectric transducer, to enhance the flow of the drug from the porous membrane into the vessel wall. The porous membrane can be left in contact for an extended time periods as the catheter contains an extra lumen to provide blood perfusion distal to the balloon. For those lesions necessitating extended drug delivery an additional lumen is provided to supply additional medicines to the lesions. As the drug(s) are passed into the tissue in way of squeezing the porous membrane, needles and the concept of diffusion are not used to deliver the drug and blood flow is not interrupted during drug delivery.

### CROSS REFERENCE TO RELATED APPLICATIONS

This application claims priority from and incorporates in its entirety, by reference, U.S. Provisional Patent Application No. 61/216,331 filed on May 15, 2009 and bearing the same title.

### FIELD OF THE INVENTION

The present invention relates to a method of targeted, or localized, delivery of therapeutic compounds through the use of shape memory devices containing pores attached to the distal end of a catheter system utilizing a pressurized pulse as a means to force fluids through small pores at high velocities into the wall of a vessel or body tissue.

### BACKGROUND OF THE INVENTION

Angioplasty and the implantation of stents have improved blood flow through occluded arteries. However, restenosis, a recurrence of blood flow restriction arising from hyperplasia, can develop after angioplasty and or stent implantation. The ability to administer therapeutic agents directly to the site of the injury or occlusion has substantial clinical benefits. For example, the drug-coated stents have shown to reduce the restenosis rate in post coronary intervention patients.

Many human diseases and abnormalities could benefit from the present invention. Vulnerable plaque that can lead to total vessel occlusion by forming massive blood clots and causing heart attacks, as well as the restenotic lesions that form from angioplasty or stenting, can potentially be reduced, or even eliminated, through the use of anti-proliferative or other pharmaceutical agents, if these agents could be administered effectively and in the proper doses. In most instances, drugs, or other therapeutic compounds, are more effective if they can be delivered directly to the affected area. The traditional systemic method of administering therapeutics involves dispensing large doses. However, as is typical with the systemic approach, only a relatively small amount of the therapeutic substance eventually reaches the affected region of the body. Therefore, a significant portion of a systemically administered therapeutic compound is essentially wasted. Additionally, the systemic use of therapeutic agents can often lead to many undesired side effects. Targeted drug delivery systems eliminate these wasteful and detrimental tendencies because the therapeutic agent is administered uniformly and in sufficient quantity to a specific locality. Drug coated stents often fail to deliver sufficient quantity of a drug due to a limited surface area available for the drug coating, and also due to the fact that the coating can unintentionally detach whenever the stent is expanded, when the stent comes in contact with hard calcific plaques, or from improper handling. It is also important that the therapeutic agents are delivered uniformly to the lesion or in increased doses to areas that are more prone to restenosis such as the ends of the stent or at calcified areas of the lesion.

Targeted drug delivery systems are particularly useful for regions of the body that are associated with rapid or turbulent fluid flow, such as the cardiovascular system. Therapeutic compounds are difficult to administer into arterial walls at sites that are occluded due to blood clots as a result of vulnerable plaque, restenosis, or other types of hyperplasia. Unless the therapeutic substance can quickly enter the arterial wall, it will be swept away by the bloodstream.

Several alternative methods have been published which attempt to administer drugs or other therapeutics into the arterial wall or other body cavities. These alternative methods can be categorized into three main types:
Drug diffusion from balloons or tubes equipped with pores or coatings: In these devices, the distal end of a catheter system is provided with a tube or a tube having a specific shape such as a curve or a coil or an inflatable balloon. This tube or balloon can either be studded with pores or alternatively be coated with a therapeutic substance. Once the tube or balloon is placed at the affected area, it is adjusted or inflated, and a drug is brought into close contact with the arterial wall via the coating or the pores. The therapeutic substance diffuses into the arterial wall at the site of the lesion or abnormality.
Drug diffusion from balloons equipped with various protrusions: In these devices, the distal end of a catheter system is provided with an inflatable balloon, which includes one or more projections. These projections do not penetrate the arterial wall. Thus, as the drug is administered from the proximal end of the catheter, it is brought in close contact with the affected area and migrates through the arterial wall by diffusion.
Drug injection from catheters or balloons equipped with needles. The distal end of a catheter system, or an inflatable balloon, is provided with one or more needles. As the needles are extended, or the balloon is inflated, the needles penetrate the arterial wall. A drug or therapeutic substance is administered from the proximal end of the catheter and travels through the lumen of the needles directly to the site of the occlusion or abnormality.

Forman in U.S. Pat. No. 5,415,636 describes a drug delivery catheter, which uses a dilation balloon, which dilates a restricted vessel. After the balloon is expanded, a drug flows from pores of the balloon. The drug can then diffuse into the vessel wall. Racchini et al. in U.S. Pat. No. 5,458,568 also describes a device that uses a porous balloon to deliver a therapeutic.

Wang et al. in U.S. Pat. No. 5,425,723 describes a drug diffusion device consisting of infusion catheter tubes which have a number of pores. The tubes are placed at the desired location within a vessel and a therapeutic is administered from the proximal end of the catheter system. The therapeutic migrates out of the pores and diffuses into the wall of the vessel.

Hanson et.al. in U.S. Pat No 5,523,092 and in U.S. Pat No 5,709,874 describes a drug delivery catheter which utilizes a substance delivery segment in the shape of a hollow coil. The coil shape exerts a force against the luminal surface enabling the substance delivery segment to become anchored at the predetermined site. Substance delivery holes are positioned on the hollow coil so that they face the luminal surface when the coil is deployed. The hollow coil can be made from Nitinol, a nickel-titanium alloy which has shape memory characteristics. When exposed to a heated fluid, the Nitinol, which has shape memory characteristics, will form into a coil. The drug travels through the holes in the coil, and, as the inventors claim, diffuses into the tissue.

Igaki et al. in U.S. Pat. No. 5,733,327 describes a drug diffusion device utilizing a balloon. The balloon is equipped with ridges, which when inflated, match the diameter of the vessel. Once the drug is administered from the proximal end of the catheter, it migrates through the pores of the balloon in order to fill tiny compartments, or reservoirs, created by the ridges of the balloon. The drug then diffuses into the arterial wall. Goodin in U.S. Pat. No. 5,397,307, Harrison et al., in U.S. Pat. No. 5,554,119, Schweich et al. in U.S. Pat. No. 5,558,642, Schweich et al. in U.S. Pat. No. 5,716,340, Levy et al. in U.S. Pat. No. 5,833,658, and Hastings et al. in Pat. No. 5,951,458 describes similar devices in which a balloon creates drug filled compartments between the balloon and the vessel wall. These types of devices can occlude the flow of blood and often require long periods for the diffusion of the therapeutic substances. In addition, the balloon may not completely adhere to the vessel wall, causing some of the administered drug to leak into the bloodstream.

Klein et al. in U.S. Pat. No. 5,810,767 describes a drug diffusion device consisting of a network of tubules that are attached to an inflatable balloon. The tubules have pores from which a therapeutic can be delivered near or adjacent to the vessel wall. However, this device blocks the flow of blood distal to the target region and can therefore cause major problems for the patient due to ischemic manifestations.

Ropiak in U.S. Pat. No. 5,843,033, and in U.S. Pat. No. 5,860,954, describe a drug diffusion device consisting of an inflatable balloon that is provided with a large number of pores. A drug is administered from the proximal end of the catheter and migrates to the distal end where it flows out of the pores of the balloon. Ungs in U.S. Pat. No. 6,149,641 describes a similar drug diffusion device consisting of a porous balloon as the means to deliver a drug to a localized region. These devices occlude the flow of blood and require long periods for the therapeutic to diffuse into the vessel wall.

Duffy et al. in U.S. Pat. No. 6,048,332 describes a drug diffusion device consisting of a balloon that is provided with a number of dimpled pores. Once the balloon is placed in the desired location via a catheter system, it is inflated, creating small chambers between the vessel wall and the pore at the bottom of the dimple. A drug is administered from the proximal end of the catheter, migrates to the distal end, and flows into the chambers created by the dimples. The drug then diffuses into the vessel wall. As with the other devices, this device will occlude the flow of blood and will require long periods for the drug to diffuse into the vessel wall.

Other inventions utilize a balloon which is coated with therapeutic substances. For example, Dror et al. in U.S. Pat. No. 5,102,402, Nicholas et al. in U.S. Pat. No. 5,588,962, Hull et al. in U.S. Pat. No. 5,893,840, and Rowe in U.S. Pat. No. 6,146,358 describes a balloon catheter that releases a drug from a coated balloon once the balloon is expanded. Similarly, Ding et al. in U.S. Pat. No. 6,364,856 and Sahatjian et al. in U.S. Pat. No. 6,409,716 describe a balloon that is coated with a polymer that contains the therapeutic compound. Once the balloon is inflated, the polymer layer presses against the vessel or tissue wall, releasing the therapeutic which eventually diffuses into the affected area. These devices have several disadvantages. For instance, coating the balloon usually involves a number of complicated processes. In addition, the coating may not sufficiently adhere to the balloon surface, causing it to unintentionally fall off the device as it is brought into position within the body especially in calcified lesions.

Palasis et al. in U.S. Pat. No. 6,369,039 describes a device that facilitates concentration-driven molecular diffusion of a therapeutic substance. The therapeutic is either held in a cavity of, or is coated onto, an inflatable balloon of an infusion catheter system. The balloon is placed at the desired location and inflated. Due to the high concentration of the therapeutic, the drug diffuses into the wall of the vessel. Richter in U.S. Pat. No. 7,048,714 describes a similar device in which a therapeutic substance is placed within the voids of a porous elastomeric material. Once the material is expanded, the therapeutic can diffuse into the vessel wall. Winkler et al. in U.S. Pat. No. 6,200,257 describes another method wherein a drug is placed into a hydrophilic substrate, which is attached to the surface of a balloon, catheter, or stent. Once this device is placed in the affected region, the drug can diffuse into the vessel or tissue wall.

Kokish et al. in U.S. Pat. No. 6,544,221 describes the diffusion of therapeutics from a system of porous balloons. Once placed at the desired location within a vessel, a therapeutic flows out from the pores of the balloon. The drug then diffuses into the vessel wall.

Lennox in U.S. Pat. No. 6,939,320 describes a drug diffusion device which consists of a therapeutic compound that is coated upon the surface of an expandable material. The drug-coated expandable device is fastened to the distal end of a catheter system and positioned to the desired location within a vessel. The device is expanded and the drug diffuses into the vessel wall. Palasis in U.S. Pat. No. 7,179,251 and Kester in U.S. Pat. No. 6,682,545 also describe coating a therapeutic agent on an expandable material, or balloon.

Rosenthal et al. in U.S. Pat. No. 7,066,904 describes a triggered drug delivery system that utilizes a hydrogel polymer as a means to administer therapeutic substances. The drug is immobilized within the hydrogel, which is then placed on the outside surface of a catheter or a balloon. A triggering event or condition contracts the hydrogel, releasing the drug to the desired body tissue. As with many of the drug delivery devices the delivery is via the diffusion process and as such, this device does not deliver a large enough dose to the affected region. Coated balloons are limited by the amount of the drug that can be adsorbed onto the surface of the balloon, and by the fact that the adsorbed compounds can be washed away by the bloodstream.

Farnan in U.S. Pat. No. 7,108,684 describes a simple drug diffusion device which utilizes an expandable balloon that is provided with a pouch. A drug is placed in the pouch and is released when the balloon expands. The released drug diffuses into the vessel wall.

Kusleika in U.S. Pat. No. 2,004,0260239 describes a drug diffusion device, which consists of a porous balloon. The balloon is expanded and conforms to the vessel wall. The drug diffuses into the vessel wall from the pores of the balloon.

Vigil et al. in U.S. Pat. Nos. 5,681,281, 6,102,904, 6,695,830, and 6,210,392 describe a drug delivery device in which the therapeutic compound is dispensed through protrusions from a balloon. Using a similar concept, Wijay in U.S. Pat. No. 5,882,332 describes a drug delivery device wherein the therapeutic compound is administered from protrusions, or bristles, that are attached to the distal end of a catheter system.

Clark et al. in U.S. Pat. No. 6,280,413 describes a drug diffusion device consisting of a number of porous longitudinal ribs that protrude from the distal end of a catheter system. Once placed at the desired location within a vessel. The ribs are expanded causing the pores to press against the wall of the vessel. The therapeutic is forced out of the pores and diffuses into the vessel wall. Even though this device does not occlude the flow of blood, the therapeutic can easily be swept away by the bloodstream.

Mizaee in U.S. Pat. No. 6,283,947 describes a drug delivery device in which the distal end of a catheter system is equipped with “injection ports.” After being placed at the proper position within a vessel, these injection ports are extended toward the vessel wall. A therapeutic substance is then administered from the proximal end of the catheter and flows through the lumens of the injection ports. The therapeutic diffuses through the arterial wall.

Hofling in U.S. Pat. No. 5,419,777 describes a drug injection device wherein the distal end of the catheter system is provided with one or more needles. The needles are contained within the device. Once they are positioned at the desired location within a vessel, the needles are extended, enabling them to penetrate the vessel wall. A therapeutic compound is then injected through the lumens of the needles.

Leonhardt in U.S. Pat. No. 5,693,029 describes a drug injection device which utilizes a needle assembly at the distal end of a catheter system. Initially, the needles are retracted until they are placed at the proper location within a vessel. Once in place, a balloon is expanded, pushing the needle assembly into the vessel wall. A therapeutic is then injected into the vessel wall through the lumens of the needles. Similarly, Haim in U.S. Pat. No. 6,254,573 and Jacobsen et al. in U.S. Pat. No. 6,302,870 have developed a drug injection devices consisting of metallic and nonmetallic needles.

Schreiner in U.S. Pat. No. 5,904,670 describes a drug injection device containing needles with shape memory characteristics. These needles can puncture the vessel wall. However, even though this device does not block the flow of blood, unlike the devices that utilize an inflatable balloon, the degree of penetration of the tissue by the needles are difficult to control as they are directed to the affected region via a catheter. In addition, there is some question as to whether enough force can be delivered to the needle assembly to enable the needles to puncture the vessel wall.

Glines et al. in U.S. Pat. No. 6,183,444 describes a drug injection catheter system which has a needle attached to a reservoir. Similarly, Flaherty et al. in U.S. Pat. No. 7,094,230 describes a drug injection device which utilizes “puncturing elements.” Also, Reed et al. in U.S. Pat. No. 6,197,013 describes a drug injection device composed of needles that are attached to an expandable balloon. Naimark et al. in U.S. Pat. No. 6,638,246 describes a similar drug injection device which is composed of numerous “micro-needles” that are on the surface of an inflatable balloon.

Wijay et al. in U.S. Pat. No. 6,997,903 describes a drug injection device that utilizes retractable needles within a catheter system. The needles puncture the vessel wall so that the therapeutic agent can be injected into the affected area. However, Goll in U.S. Pat. No. 6,344,027 describes a “needle-less” injection apparatus wherein a therapeutic is forced through a nozzle which is in contact with the vessel wall. As with any device that uses needles to inject a drug, these devices puncture the vessel wall and can therefore potentially cause damage to the vessel tissue.

Ahem et al. in U.S. Pat. No. 6,251,418 describes another drug delivery concept consisting of a method of implanting pellets containing a therapeutic substance.

Don Michael in U.S. Pat. No. 5,176,638 describes a regional perfusion catheter with drug delivery into a minienvironment having single or multiple balloon to isolate the segment treated. This concept as those of others depend on the diffusion of the drug into the tissue.

Crocket et al. in U.S. Pat. No. 5,421,826 describe a drug delivery and dilatation catheter having a reinforced perfusion lumen. The balloon has two layers and the drug is introduced to the space between outer and inner balloon and diffuses from this cavity. This concept although is theoretically appealing the construction of the device is not economical or workable.

Sahota in U.S. Pat. No. 5,160,321 describes a catheter having an extra lumen that permits blood flow distally during balloon dilatation but does not provide a means for injecting drug into the surrounding vascular tissue.

Leone in U.S. Pat. No. 5,674,198 describes a dual balloon catheter inflated independently and having an infusion lumen that opens between the two balloons to deliver the drug. The concept also uses diffusion means to introduce the drug to the blood vessel wall, which is a very slow process.

Sahota in U.S. Pat. No. 5,951,514 describes a catheter having multiple balloons and a drug Infusion-port between adjacent balloons while maintaining blood flow during balloon inflation.

Houser in U.S. Pat. No. 6,632,196 describes means of delivering a drug using multilayered balloon similar to those of Leone. The disadvantage is the presence of multilayer and multi lumens to inflate such numerous balloons.

Palasis in U.S. Pat. No. 7,060,051 describes a multi balloon catheter having an infusion lumen in between exactly like Sahota having hydogel-coated balloon therein.

Klien in U.S. Pat. No. 5,863,284 describes a catheter having a balloon containing radiation strips attached to the outer wall of the balloon. On inflation the strips are in contact with the blood vessel delivering radiation to the vessel.

Simpson in U.S. Pat. No. 5,462,529 describes a drug delivery catheter having two balloons that are independent of each other thereby enabling adjustment of the distance between them. The drug is infused between the balloons to the desired segment of the artery. Formen et al. in U.S. Pat. No. 5,772,632 describe a combination of dilatation catheter and double balloon catheter to deliver drugs to a target area.

O'Brien et al. in U.S. Pat. No. 6,575,932 describe a double balloon catheter with capability to adjust the distance between the balloons, like the Simpson in U.S. Pat. No. 5,462,529. Forman in U.S. Pat. No. 6,997,898 describe a multi-balloon catheter having drug infusion lumens between the balloons, similar to Sahota in U.S. Pat. No. 5,951,514.

### SUMMARY OF THE INVENTION

The present invention describes the creation of a unique catheter, which will deliver therapeutic compounds to a localized area within the cardiovascular system, such as coronary arteries as well as other narrow conduits, or cavities of the body, such as arteries, the bile duct, bronchi, urethra, ureter, heart, and bladder. Drugs or other therapeutic compounds will be forced through a vessel or tissue wall due to the inflation of a balloon and by maintenance of the drug containing porous member in contact with the blood vessel wall for a period of time. To achieve this drug is carried on a thin distensible porous membrane that is affixed on to an expandable member such as a dilatation balloon. On expansion and in contact of the wall of the vessel therein the drug is forced out of the porous membrane into the wall of the vessel due to the pressure exerted by the balloon as well as due to contact of the drug carrying membrane with the blood vessel wall. Additionally by using an ultrasound or sub—ultrasound vibration imparted using piezoelectric or similar methods on to the porous membrane, the penetration and transportation of the drug into the vessel wall therein can be enhanced. Additional drug may be provided by way of an additional lumen provided in the catheter for this purpose in situations where continuous supply of drug over a long period is necessary. The balloon will be chosen carefully so that the outer diameter of the balloon and the porous membrane is such that there will be no over inflation of the blood vessel. The catheter can be designed to work as an over the wire (OTW) or rapid exchange (RX) catheter while accomplishing the same objective. In situations where blood flow distal to the balloon is required, such as in coronary arteries, an additional lumen by passing the balloon is provided for this purpose. In most cases this lumen is also the guide wire lumen, whereby the guide wire is partially withdrawn to enable the flow of blood through the said lumen distally to the lesion in the artery. It is often necessary to re-advance the guide wire distal to the lesion. And in other designs this is not possible or an additional lumen is provided for this purpose. In the present design a special feature is incorporated into this invention that will enable the operator to re-advance the guide wire after the drug therapy is completed in order to withdraw or relocate the catheter. This feature is not available in the other perfusion catheters in the literature or in prior art. In an alternate arrangement to the present design an additional lumen can also be provided so that additional drug can be supplied into the porous membrane via this additional lumen. This feature will be useful when treating long lesions without having to remove the catheter to reload the porous membrane with the therapeutic drug.

The present device has many advantages over systemic or other targeted drug delivery devices discussed earlier in that it (a) does not puncture the vessel or tissue wall, as is the case with catheters containing needles, and (b) it does not require diffusion, which is a very slow process, as a means for drug absorption into the affected area, such as the case in drug coated balloons. The wall of a vessel could become damaged if it is punctured by needles or sharp protrusions causing additional injury resulting in inflammation. Additionally, drug delivery devices that use diffusion as the means to administer therapeutic agents into tissues are inefficient because the therapeutic agent may leak into the bloodstream and get washed away, or by the fact that only a small, insufficient amount of the drug successfully diffuses into the affected target area or lesion.

The present invention has substantial improvement over many multi-balloon catheters in prior art having an infusion lumen therebetween the balloons. These devices with multiple balloons while isolating the treatment area are unable to force the drug into the vessel wall without over inflation of the blood vessel that is very detrimental. Moreover, the space between the balloons is typically filled with blood and therefore the drug will mix with blood diluting the effects therefore. In those inventions where the balloons are inflated separately to avoid this problem, the size of the catheter, i.e. the profile increases substantially to provide the additional lumen for the second balloon inflation. Those systems having drugs attached to strips on the balloon and other means lack coverage as the drug is not supplied uniformly over the balloon surface.

Those devices where the drug is coated on the balloon using a polymer have two distinct disadvantages. First, a binder, typically a polymer, bonds the drug to the balloon wall and hence does not readily release the drug in order to enter the vessel wall and secondly the amount of the drug that can be bonded to the balloon wall is limited and therefore the total amount of the drug delivered is often insufficient.

Another advantage of the present device over the coated balloons and coated stents is that the user can chose the type of drug that is administered based on the lesion that is infused with the drug. Combination of drugs can also be used and in fact tandem treatment is also possible with the present invention, whereby one drug is infused first and the second drug is infused afterwards. As such, in this invention, unlike the coated balloon and coated stent the user has the choice of the drug to be infused.

The present invention is very simple and easy to make, reducing the need for complicated production and handling procedures as in the case of catheters containing needles, catheters using heated fluids to restore predetermined shapes, such as coils, or catheters using coated balloon devices or coated stent devices wherein the limited drug coating may dissolve quickly or detach due to mechanical handling or can be readily washed away by the blood stream.

The thin porous membrane is simply attached to the angioplasty balloon by various attachment means known in the industry, such as bonding, crimping etc. The porous membrane is then soaked with the drug or a solution containing the drug and let dry. A protecting cap can then be placed over the porous membrane to protect the porous membrane until the device is used. When used the cap is removed, the catheter is advanced to the desired location and inflated. The drug is forced out of the porous membrane to enter into the vessel wall. If additional drug infusion is deemed necessary, the catheter can be withdrawn and the porous membrane can be resoaked in the drug after squeezing out any blood from the membrane and the catheter is reintroduced to repeat the process. In addition, the device can be supplied without a drug incorporated and the user can soak the tip of the catheter in the drug of his choosing before introducing the catheter into the lesion for treatment.

The present device also incorporates a perfusion lumen that will allow blood flow during this procedure so that the balloon can be in place inflated for a long period of time without having to deflate due to any ischemic manifestations or discomfort to the patient. Additionally the ultrasonic energy and the non-ultrasonic energy enhance the process by which the drug is introduced into the vessel wall. The ultrasonic module can be within the balloon or can be attached to the proximal hub of the balloon inflation lumen.

Thus, unlike many other drug delivery devices, the present invention delivers therapeutic compounds to its target without obstructing blood flow, or other biological processes, within the vessel or tissue. There is no limit to the amount of the drug or the biological agent that can be injected as the porous membrane can absorb very large quantities of drug and the present invention also allows the operator to deliver varying quantities of the drug to different areas by properly positioning the porous membrane in the inflated balloon in those areas typically requiring more medication, so that more of the drug can be delivered to areas that require more treatment.

The catheter is a balloon catheter having a distal and a proximal end where the balloon is attached to the distal end of the catheter. A dedicated lumen is provided to inflate and deflate the balloon. Both the OTW and the RX types of the balloon catheter is provided with a guide wire lumen in order to thread the catheter into the vasculature. A luer-hub is provided at the proximal end so as to connect the catheter to an inflation device, for balloon inflation. The catheter shaft can be made from any of the plastics commonly used for catheter shaft, such as Nylon, Polyethylene, Polyurethane or a Teflon coated hypo tube. The balloon can be made from Nylon, Co-polymers of Nylon, PET, polyurethane and other similar material. The distensible and expansible porous tube can be made from any material that is porous and expansible, such as Polyurethane, Polyethylene, Nylon, Silicone, PVC, Polyvinyl-pyrrolidone, PMMA, Cellulose, porous Cellulose, Porous Cellulose Acetate, Porous Cellulose Nitrate etc. The porous tube can be made from knitted fiber or woven fiber where the fiber is naturally occurring such as silk, cotton or manmade such as polyester, nylon, polypropylene or expanded PTFE. The knit size can be varied to achieve the desired porosity and fluid holding capacity to suit the application. The porous tube can also be made from collagen and spun collagen. The expansible porous tube is attached by any one of the attachment means such as adhesive bonding, crimping, winding and other techniques.

The drug can be soaked into the balloon by immersing a balloon into a solution of the drug with a known concentration of the drug. The balloon is subsequently dried in air or vacuum so that the drug stays in the pores until it is squeezed out during the deployment. Alternatively, the catheter can be sterilized and provided as-is to the user who would immerse the balloon in a solution of the drug immediately before it is used.

### BRIEF DESCRIPTION OF THE DRAWINGS

FIG. 1 shows one embodiment of the proposed invention with the Over The Wire balloon catheter (angioplasty balloon catheter) having a folded balloon and the distensible porous tube affixed over it.

FIG. 2 shows one embodiment of the proposed invention with the Over The Wire balloon catheter having the balloon inflated and the distensible porous tube also expanded along with the balloon.

FIG. 3 shows the said balloon catheter having the balloon inflated inside an artery and the distensible porous tube also expanded along with the balloon, in contact with the artery wall and delivering drugs to the arterial wall.

FIG. 4 shows an alternate balloon catheter design, the Rapid Exchange design (RX) having the balloon inflated and the distensible porous tube also expanded along with the balloon. The additional lumen provides blood flow distal to the balloon when the guide wire is partially and sufficiently withdrawn.

FIG. 5 shows the said RX balloon catheter having the balloon inflated and the distensible porous tube also expanded along with the balloon. The guide wire is withdrawn so as to allow blood to allow to flow into the perfusion lumen and distal to the balloon.

FIG. 6 shows a diagrammatic detail of the ports designed for blood to flow into the perfusion lumen but the lip that is bent inwards in to the port does not allow the guide wire to exit the perfusion ports during guide wire advancement.

FIG. 7 shows a diagrammatic detail of the catheter similar to the catheter shown in FIG. 5 with an additional lumen to continuously provide drugs to the expansible porous membrane.

### DETAILED DESCRIPTION OF THE PREFERRED EMBODIMENT

The object of this invention is to provide an effective means of delivering various anti-proliferative drugs or other therapeutic or biologically active agents into a targeted area without damaging or puncturing the blood vessel or tissue wall. The preferred embodiment consists of a delivery catheter, which is a long slender tube that is typically 30 to 150 centimeters long, with a drug delivery module located at its distal end. A lumen is provided to enable the catheter to be advanced over a guide wire, either as an over The Wire balloon catheter or as a rapid Exchange balloon catheter, which helps position the delivery module to the proper location.

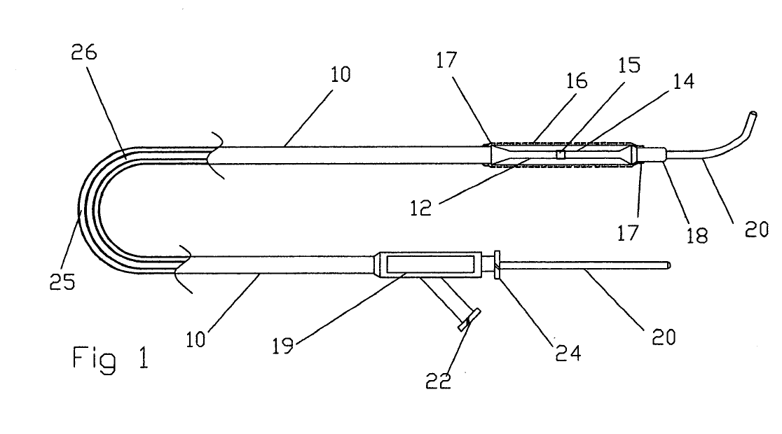

The catheter in FIG. 1 is a balloon catheter (10) having a distal portion and a proximal portion wherein the distal portion has a balloon (12) attached to it and the proximal portion has a hub (19) attached so that the balloon can be inflated via the inflation lumen (25) which is in fluid communication with the side arm (22) of the hub (19). An additional lumen (26) is provided connected to the hub (19) so as to advance a guide wire (20) through the luer (24) for positioning the balloon (12) at the lesion. The inflation lumen (25) is in fluid communication with the luer port (22) in the catheter hub (19). These functions are accomplished by the co-axial design well known in the art where the balloon is inflated via the lumen formed by the space between the two co-axial tubes. The balloon (12) is attached to the catheter tube by heat bonding or other means. Radiopaque marker(s) (15) is placed within or outside the balloon area so that the catheter and balloon location is visible under fluoroscopy. The catheter has an atraumatic tip (18) at its distal end and the guide wire (20) exits the catheter at this location. The catheter as shown in FIG. 1 is a co-axial balloon catheter. The balloon of the catheter is folded over the catheter inner tube. Balloon (12) can be folded to a bi-fold, a tri-fold or multi-fold as shown by numeral (14) without departing from the present invention. The expansible sleeve member (16) is placed over the balloon after folding the balloon and is secured distal to the balloon and proximal to the balloon at the balloon shoulders (17). The expansible member is in tube form, although other geometric forms may also be used. The attachment of the expansible member can be accomplished by adhesive bonding, crimping a metal ring or by winding a thread over the expansible member. In addition to a metal crimp and/or wound thread, adhesives can also be used to further secure the expansible member to the balloon shoulder. The expansible member is porous and is capable of absorbing fluids, fluids containing bioactive agents such as anti-proliferative drugs and is capable of expanding radially when the underlying balloon is inflated. The pore size can vary from nanometers to micrometers and can be open cell as well as closed cell. Many materials such as silicone, polyurethane, polyethylene, and cellulose, silk, knitted or woven tubes made from polyester, nylon or silk etc can be used as a porous member (16).

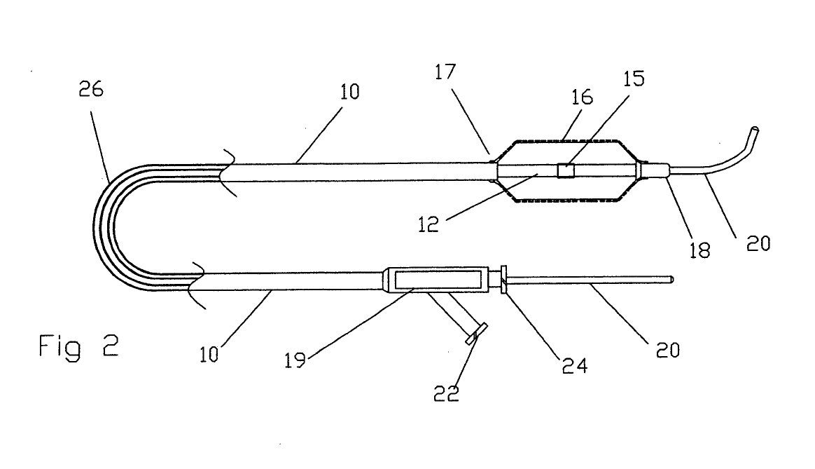

FIG. 2 shows the above-described catheter where the balloon is inflated with a suitable fluid. The fluid mostly used to inflate the balloon is a diluted radio-contrast solution. To inflate the balloon, an inflation device (not shown) is connected to the luer port (22) and the contrast solution is introduced and the inflation pressure is adjusted until the desired pressure, which in turn relates to the desired diameter of the balloon, is achieved. When the balloon is inflated as described above, the distensible member (16) also is stretched and its diameter will grow squeezing the drug that is trapped in the pores until the drug is pushed out of the expansible sleeve into the surrounding tissue.

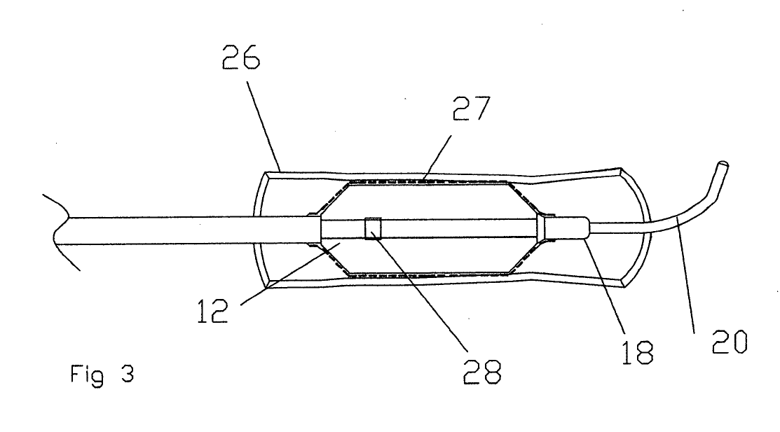

FIG. 3 shows this diagrammatically; wherein the inflated balloon (12) and the expansible sleeve member (16) carrying the drug is in contact with the blood vessel (26) at the lesion (27). The balloon and the expansible sleeve (porous member) carrying the drug is made to contact with the vessel wall as long as possible to ensure that the drug is effectively transferred to the vessel wall. Piezoelectric transducer (28) can be activated via the wires (29) to create further transport of the drug from the porous membrane to the vessel wall.

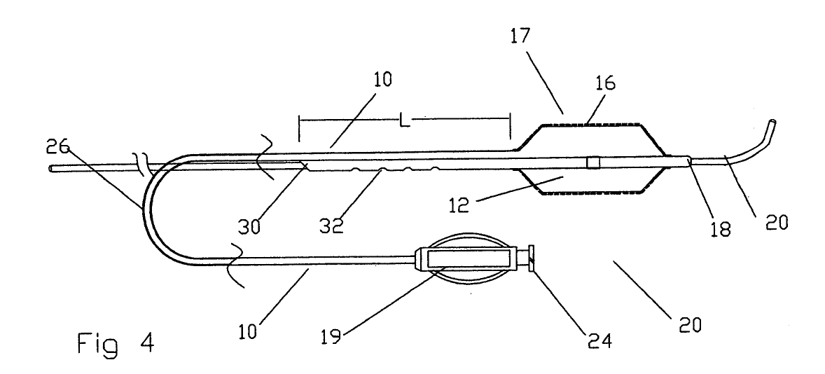

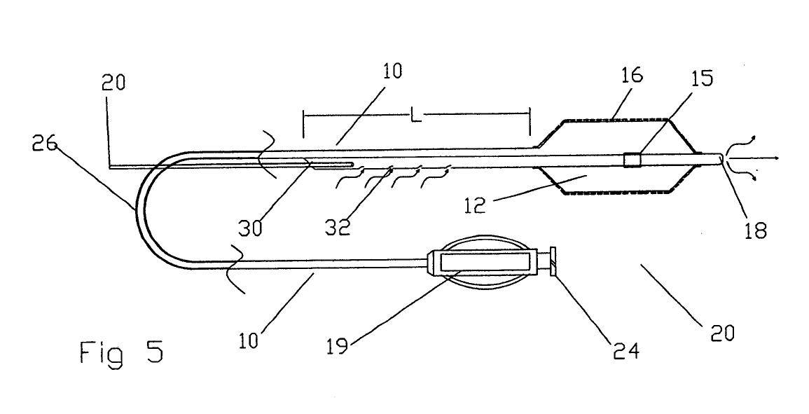

FIG. 4 shows diagrammatically an alternate design for the same purpose wherein blood flow distal to the balloon is maintained even after balloon inflation. This is accomplished by the additional lumen (30) provided for this purpose. The same lumen is also used as the guide wire lumen whereby the guide wire (20) is advanced distal to the balloon for the purpose of threading the catheter to the desired location. After positioning the balloon at the lesion, before balloon inflation the guide wire is withdrawn sufficiently to expose the multiple side ports (32) in the said guide wire lumen as shown in FIG. 5. Blood enters the lumen at these side ports (32) and flows distal to the balloon to the distal portion of the vasculature. Therefore during balloon inflation, blood is continually supplied distal to the lesion through this lumen (30) keeping the distal myocardium perfused and the patient free of pain and without any electro-cardiologic episodes. The length “L” of the perfusion lumen is typically 25 cm however depending on the application other lengths can also be used effectively.

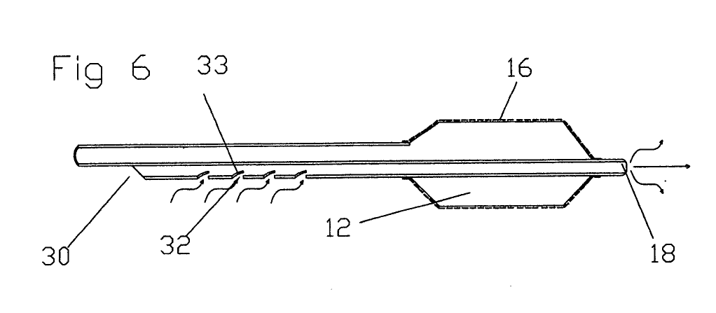

FIG. 6 diagrammatically shows the feature in the ports (32) that allows blood to enter the lumen (30) so that blood from multiple ports will travel distal through the lumen (30) once the guide wire is partially withdrawn to clear the multiple ports (32). However it is often necessary to re-advance the guide wire. But it is difficult to re-advance the guide wire beyond the ports (32) as the guide wire tip gets entangled in the ports (32) in the catheter designs that are in use at present. This is avoided in the design in FIG. 6. The ports are made such that a flap or lip (33) in the ports is bent inwards at the ports (32). Thus blood can enter the lumen through the port (32). When the guide wire is re-advanced in the lumen (30) the guide wire tip jumps over the flap (33) without exiting the port (32). Thus the guide wire (20) can be re-advanced distal to the balloon in order to reposition the catheter or withdraw the catheter entirely so as to re-soak the catheter with the appropriate drug for additional treatment of the lesion.

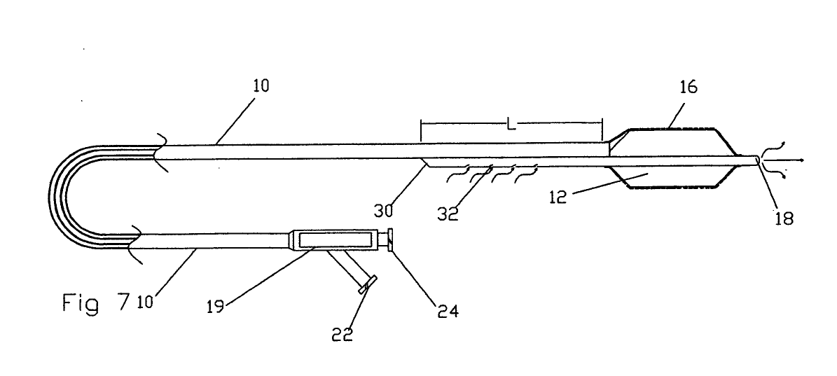

FIG. 7 provides an alternate design for the above-mentioned catheter, where an additional lumen is provided to continuously provide medicines and bio-active agents when the balloon is inflated to lesions requiring extended drug delivery. The catheter in FIG. 7 has a catheter body, which is either co-axial or double lumen. One of the lumens is in fluid communication with the balloon and is for the purpose of inflating the balloon. The other lumen that is in fluid communication with the side arm of the hub (19) via the luer (22) opens to the porous expansible member 16 at or near the proximal shoulder of the balloon. The catheter in FIG. 7 also has a perfusion lumen (30) similar to the catheter in FIG. 3 so that blood can flow distal to the balloon once the balloon is inflated, thereby providing blood flow to the distal myocardium.

We claim:

1. A catheter for a patient conduit or cavity having a wall, comprising:
an elongated body comprising at least one lumen;
a balloon in fluid communication with said lumen;
a discrete covering capable of storing at least one drug that is mounted over said balloon and said covering selectively releasing said drug when compressed against the wall of the conduit or cavity due to balloon inflation through said lumen.

2. The catheter of claim 1, wherein:
said covering has shape memory.

3. The catheter of claim 1, wherein:
said covering is porous.

4. The catheter of claim 1, wherein:
said covering retains said drug in its pore structure.

5. The catheter of claim 4, wherein:
said covering is loaded with said drug by immersion.

6. The catheter of claim 1, wherein:
said covering is reusable after removal for application of additional drug prior to reinsertion.

7. The catheter of claim 1, wherein:
said covering is secured to said balloon or said body.

8. The catheter of claim 1, wherein:
said at least one lumen comprises a balloon lumen for selective balloon inflation and a bypass lumen to allow flow past the balloon when against a wall.

9. The catheter of claim 8, wherein:
said bypass lumen extends the substantial length of said body and through said balloon and has at least one inlet port proximally of said balloon and at least one discharge port distally of said balloon.

10. The catheter of claim 9, wherein:
said inlet port is shielded to allow a distally advancing guide wire to pass over said inlet port.

11. The catheter of claim 10, wherein:
said shielding comprises a portion of said bypass lumen wall pushed in on an end closer to said balloon.

12. The catheter of claim 8, wherein:
said balloon and said bypass lumens are either coaxial or side by side.

13. The catheter of claim 8, further comprising:
a drug perfusion lumen through said body to reach said covering to allow delivery of drug to said covering when said covering is in said conduit or cavity.

14. The catheter of claim 13, wherein:
said drug perfusion lumen extends through said balloon.

15. The catheter of claim 1, wherein:
a piezoelectric device in said balloon to vibrate the fluid in said balloon for enhanced delivery of drug from said covering.

16. The catheter of claim 1, further comprising:
a drug perfusion lumen through said body to reach said covering to allow delivery of drug to said covering when said covering is in said conduit or cavity.

17. A catheter for a patient conduit or cavity having a wall, comprising:
an elongated body comprising a balloon lumen extending to a balloon located near a distal end of said body and a bypass lumen;
a balloon in fluid communication with said balloon lumen;
said bypass lumen comprising at least one shielded inlet located proximally to said balloon.

18. The catheter of claim 17, wherein:
said shielding comprises a portion of said bypass lumen wall pushed in on an end closer to said balloon.

19. The catheter of claim 17, wherein:
said at least one inlet comprises a plurality of inlets.

20. The catheter of claim 17, further comprising:
a guide wire;
said bypass lumen extending the substantial length of said body;
said shielding deflecting a guide wire that advanced distally toward said balloon and over said inlet away from said inlet.

21. The catheter of claim 20, further comprising:
a discrete covering capable of storing at least one drug that is mounted over said balloon and said covering selectively releasing said drug when compressed against the wall of the conduit or cavity due to balloon inflation through said balloon lumen.
